(19) United States
(12) Patent Application Publication
(10) Pub. No.: US 2024/0389940 A1
(43) Pub. Date: Nov. 28, 2024

(54) Hydration Monitoring Device and System

(71) Applicant: Wilda Diaz, Maywood, NJ (US)

(72) Inventors: Wilda Diaz, Maywood, NJ (US); Michael Marks, Orleans, MA (US)

(73) Assignee: Wilda Diaz, Maywood, NJ (US)

(21) Appl. No.: 18/674,572

(22) Filed: May 24, 2024

Related U.S. Application Data
(60) Provisional application No. 63/468,793, filed on May 25, 2023.

(51) Int. Cl. A61B 5/00

(52) U.S. Cl. CPC A61B 5/4875; A61B 5/6887; A61B 5/742

(57) ABSTRACT

A hydration monitoring device and system is provided. The hydration monitoring device includes housing having a base with a receptacle for holding a water bottle, a weight sensor to detect changes in the water bottle's weight, and a transceiver for connectivity with an application on an electronic device. A display is disposed on an exterior of the housing and a speaker for delivering visual and audible notifications. The display is a touchscreen to provide a user interface for allowing the user to customize notifications and settings. The weight sensor tracks water consumption, and the system calculates a desired hydration rate based on user data and activity levels. The device and system provide each provide visual and auditory prompts to the user when water consumption falls below the target rate, ensuring adequate hydration. This system facilitates real-time monitoring and encourages consistent water intake, enhancing user health and well-being.

### CROSS REFERENCE TO RELATED APPLICATION

This application claims the benefit of pending U.S. Provisional Application No. 63/468,793 filed on May 25, 2023; the above identified patent applications are herein incorporated by reference in its entirety.

### BACKGROUND OF THE INVENTION

The present invention relates to a hydration monitoring device and system. More specifically, the present invention provides a housing for measuring water consumption in a water bottle and an integrated system that tracks the water consumption, wherein the system prompts a user to maintain adequate hydration through visual and auditory notifications.

Adequate hydration is essential for maintaining overall health and well-being. Water consumption is the primary means of hydration, and people often carry water bottles to ensure they can hydrate on the go. Despite the widespread availability of reusable and disposable water bottles, ensuring consistent water intake remains a challenge for many individuals. Existing water bottles may have markings to indicate fluid levels, but they do not provide real-time monitoring or reminders to encourage regular consumption.

Various health tracking devices and applications, such as fitness trackers and smartwatches, include hydration tracking features, which allow users to manually log their water intake. However, these solutions typically rely on users to manually input their water consumption data. This manual entry process can be inconvenient, leading to inaccurate tracking and reduced user compliance.

Some intelligent water bottles incorporate digital displays to show the amount of water consumed, and they may even sync with smartphone apps. However, these bottles often have limitations, such as the need for proprietary bottles, limited compatibility with other containers, and inadequate reminder systems. These smart bottles fail to provide timely notifications or personalized prompts based on user activity levels and environmental factors.

The primary shortcomings of existing hydration tracking methods include the need for manual data entry, lack of real-time monitoring, and insufficient reminder systems. Users may forget to log their water intake or become desensitized to generic reminders, resulting in suboptimal hydration. Therefore, there exists a need for a hydration monitoring device and system configured to seamlessly measure water consumption and send notifications to the user.

In light of the devices disclosed in the known art, it is submitted that the present invention substantially diverges in design elements and methods from the known art and consequently it is clear that there is a need in the art for a hydration monitoring device and system. In this regard the instant invention substantially fulfills these needs.

### SUMMARY OF THE INVENTION

In view of the foregoing disadvantages inherent in the known types of hydration monitoring devices and systems now present in the known art, the present invention provides a new hydration monitoring device having an integrated system that tracks water consumption and prompts users to maintain adequate hydration through visual and auditory notifications.

It is an objective of the present invention to provide a hydration monitoring device comprising housing having a base with a receptacle to hold a bottle, a weight sensor to measure water consumption, a thermometer to track ambient temperature, and a Bluetooth module for seamless connectivity with an application on an electronic device. A touch screen display and speaker are disposed on the housing and configured to deliver personalized visual and auditory notifications, ensuring users are reminded to hydrate according to their individual needs and activity levels.

It is an objective of the present invention to provide a hydration monitoring device wherein the receptacle is cylindrical in shape and comprises a tapered upper end to receive a wide range of water bottle sizes and designs, enhancing user convenience and flexibility.

It is an objective of the present invention to provide a hydration monitoring system wherein the application is configured track a user's daily water consumption by measuring the weight of water being removed from a water container over a given period of time. The water container is stored in the receptacle and placed on the base. When the water bottle is placed into the holder, the holder weight of the water bottle is recorded and sends the data to an application on a user's electronic device, such as a smartphone.

It is therefore an object of the present invention to provide a new and improved hydration monitoring device and system that has all of the advantages of the known art and none of the disadvantages.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a front perspective view of an embodiment of the hydration monitoring device and system.

FIG. 2 shows an exploded view of an embodiment of the hydration monitoring device and system.

FIG. 3 shows a rear perspective view of an embodiment of the hydration monitoring device and system.

FIG. 4 shows a block diagram of an embodiment of the hydration monitoring device.

### DETAILED DESCRIPTION OF THE INVENTION

Reference is made herein to the attached drawings. Like reference numerals are used throughout the drawings to depict like or similar elements of the system. For the purpose of presenting a brief and clear description of the present invention, the embodiment discussed will be used for housing for measuring water consumption in a water bottle and providing prompts to a user to maintain adequate hydration through visual and auditory notifications. The figures are intended for representative purposes only and should not be considered to be limiting in any respect. Furthermore, the described features, structures, or characteristics may be combined in any suitable manner in one or more embodiments. In the following description, numerous specific details are provided to give a thorough understanding of embodiments.

Reference will now be made in detail to the exemplary embodiment (s) of the invention. References to “one embodiment,” “at least one embodiment,” “an embodiment,” “one example,” “an example,” “for example,” and so on indicate that the embodiment(s) or example(s) may include a feature, structure, characteristic, property, element, or limitation but that not every embodiment or example necessarily includes that feature, structure, characteristic, property, element, or limitation. Further, repeated use of the phrase “in an embodiment,” “first embodiment,” “second embodiment,” or “third embodiment” does not necessarily refer to the same embodiment.

As used herein, “computer-readable medium” or “memory” excludes any transitory signals, but includes any non-transitory data storage circuitry, e.g., buffers, cache, and queues, within transceivers of transitory signals. As used herein, “logic” refers to (i) logic implemented as computer instructions and/or data within one or more computer processes and/or (ii) logic implemented in electronic circuitry.

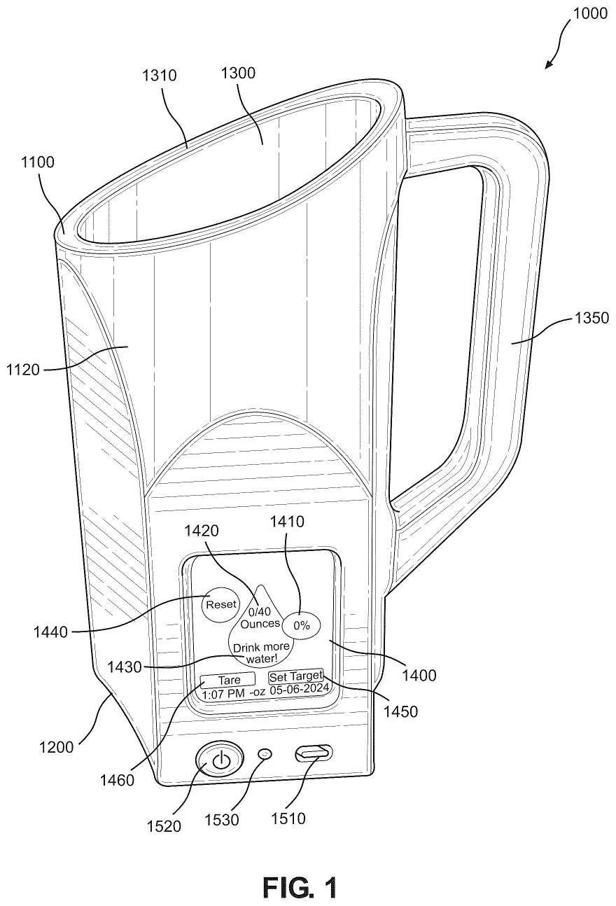

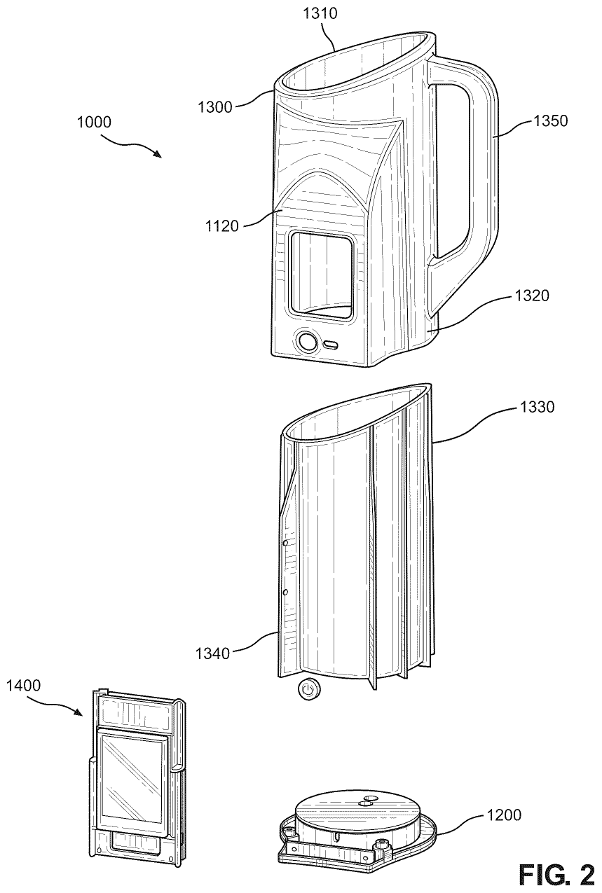

Referring to FIGS. 1 and 2, there is shown a front perspective view and an exploded view of an embodiment of the hydration monitoring device and system, respectively. The hydration monitoring device and system 1000 comprise a housing 1100 having a base 1200 with a receptacle 1300 extending from the base. An open upper end of the receptacle 1300 is adapted to receive a water bottle therein and the base is configured to weigh the water bottle, via a weight sensor. In the illustrated embodiment, the housing 1100 comprises a substantially cylindrical shape and a tapered upper end 1310. The cylindrical shape and the tapered opening allow for the accommodation of various bottle sizes and shapes to be received within the housing, ensuring that the bottles can be easily inserted, as well as easily removed from the housing 1100. In alternate embodiments, the housing comprises any suitable shape. The open upper end of the housing 1100 is designed to receive a standard-sized water bottle, with a diameter up to 4 inches. This dimension is sufficient to accommodate most commercially available water bottles, both reusable and disposable, ensuring versatility and user convenience. In alternate embodiments, the open upper end is configured to receive a bottle having a diameter larger than 4 inches.

The receptacle 1300 of the hydration monitoring device, as depicted in FIG. 2, is constructed comprises two concentric walls: an outer wall 1320 and an inner wall 1330, creating a space within the interior of the walls 1320, 1330. Braces 1340 extend vertically between the upper and lower ends of the receptacle 1300, providing structural support and maintaining the spacing between the walls 1320, 1330. Within this interior space, various electronic components are housed, including the display screen 1400. The design allows the display screen 1400 to be securely mounted within the receptacle 1300 without protruding, protecting it from external damage and ensuring it remains easily visible to the user. The double-wall construction assists in isolating the electronic components from the water bottle, preventing any potential damage due to moisture or accidental spills. In some embodiments, the base 1200 is separable from the receptacle 1300, wherein the receptacle forms a sleeve when detached from the base. In this way, the receptacle and the base can be separated and cleaned, if needed. In other embodiments, the base is permanently secured to the receptacle. In the illustrated embodiment, a portion of the base sits within the receptacle when the housing is formed.

A handle 1350 extends from an exterior of the receptacle 1300 to allow the housing 1100 to be grasped and easily moved. The handle 1350 is positioned on the side of the housing, extending vertically from the midsection to the upper section of the device. It is integrated into the housing in such a way that it provides a firm and comfortable grip for the user. The handle's placement ensures that the device can be easily carried and maneuvered, whether the user is holding it directly or using it to insert or remove the water bottle. In alternate embodiments, the housing does not include a handle.

The display 1400 is accessible through an exterior 1120 of the housing 1100 and is adapted to present information to a user. In the illustrated embodiment, the display 1400 is a touch screen. The touch screen is adapted to manage settings of the device and present messages to the user. In the illustrated embodiment, the display 1400 is disposed on the front lower section of the housing 1100. In the shown embodiment, the housing 1100 comprises a flat exterior sidewall 1120 to accommodate the display 1400. This flat surface ensures that the display screen is securely mounted and clearly visible to the user, providing an optimal viewing angle for reading information and interacting with the touch screen interface.

The display 1400 is adapted to show a current water consumption 1410 in ounces, a set hydration target 1420, and reminders 1430 such as “Drink more water!”. Additionally, the display 1400 includes interactive buttons for resetting data 1440, setting the target 1450, and taring 1460 the scale. The user is able to set the hydration target 1420 based on their hydration goal for a specific period of time, such as a daily target. In the illustrated embodiment, the user can enter their hydration goals and adjust the time settings. The interface also provides real-time updates on the user's progress towards their hydration target, displaying the amount of water consumed and the remaining amount needed to meet the goal. The display's 1400 interface is designed to be user-friendly, ensuring that users can easily monitor their hydration and adjust settings as needed.

If a hydration target 1420 is not set by the user, the system is configured to calculate a default hydration target that is determined by an ambient temperature, detected by a temperature sensor disposed within the housing. In some embodiments, the display 1400 shows the temperature as detected by the temperature sensor.

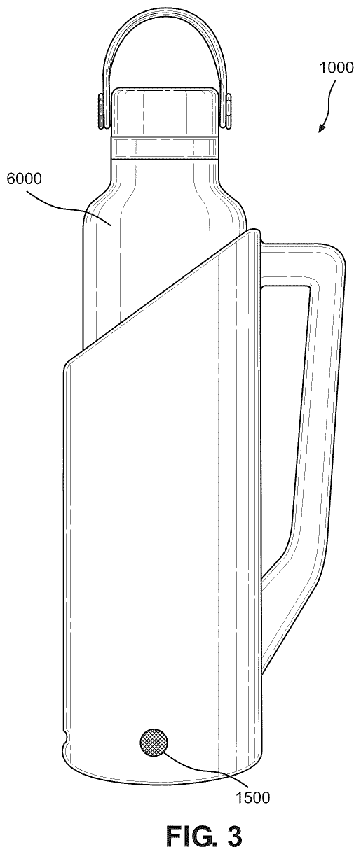

In some embodiments, as shown in FIG. 3, the hydration monitoring device and system 1000 comprise a speaker 1500 disposed on the housing 1100 for delivering audible notifications to the user. In this way, the display and the speaker are configured to provide both visual and auditory prompts, respectively, to the user when the rate of water consumption falls below a predetermined target.

The hydration monitoring device and system further comprise a power source for powering the weight sensor and display 1400. In some embodiments, the power source is a rechargeable battery and a charging port 1510 is disposed on the housing for charging the battery. This input typically supports USB-C connectivity, allowing for fast and efficient charging. An on/off button 1520 is situated on the lower front part of the housing 1100, just below the display 1400. The button 1520 is used to power the device on and off, allowing the user to activate or deactivate the hydration monitoring functionality as required. The power input is located near the on/off button, providing a convenient point for recharging the device's internal battery.

In some embodiments, an indicator light 1530 is positioned between the on/off button 1520 and the charging port 1510. The indicator light 1530 is configured to illuminate to indicate the charging status of the device and providing feedback on the Bluetooth connectivity. When the device is charging, the light may display a steady or blinking pattern to indicate the charging progress. When the device is connected to an application via Bluetooth, the indicator light confirms the connection status.

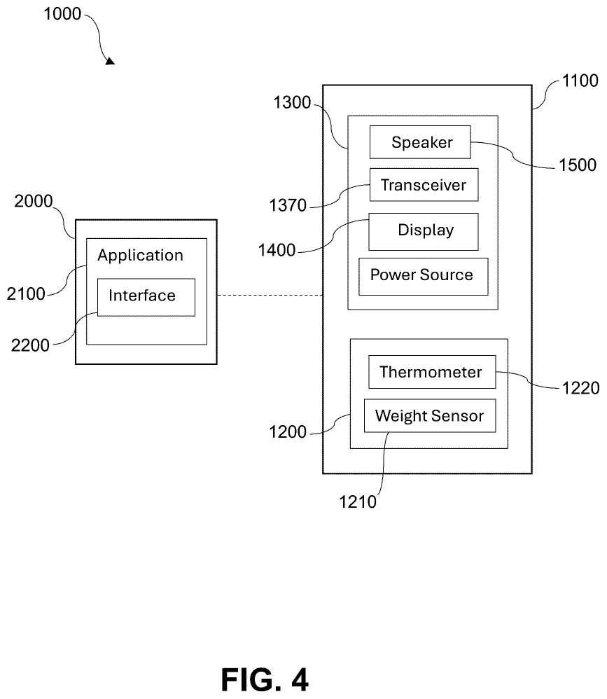

Referring now to FIGS. 3 and 4, there is shown a rear perspective view of an embodiment of the hydration monitoring device and system and a block diagram of an embodiment of the hydration monitoring device and system, respectively. The weight sensor 1210 is disposed in the base 1200 and is configured to detect a weight of a water bottle 6000 placed on the base of the housing 1100. The weight sensor tracks water consumption by measuring a change in weight of the water bottle 6000.

In some embodiments, a thermometer 1220 is integrated into the housing to measure the ambient air temperature. The thermometer 1220 is positioned near the base, this placement ensures that the thermometer can reliably capture the surrounding temperature without interference from the internal heat generated by the device's electronic components. The purpose of the thermometer is to provide environmental data that affects the user's hydration needs. By measuring the ambient temperature, the device can adjust hydration targets and reminders to account for variations in temperature that influence the rate of water loss through perspiration. This integration allows the hydration monitoring system to offer more personalized and accurate hydration recommendations, enhancing the overall effectiveness of the device in promoting adequate hydration based on real-time environmental conditions.

A transceiver 1370 is disposed within the housing for wireless communication with an application 2100 on an electronic device 2000, such as a smart device. The application 2100 is configured to calculate a desired hydration rate based on user profile data and activity levels. An interface 2200 on the electronic device is adapted to receive a user weight and user activity level to calculate a personalized hydration target. The hydration monitoring device 1000 utilizes a Bluetooth module as the transceiver to wirelessly transmit data detected by the weight sensor, display screen input, and thermometer to the application on the electronic device 2000. This Bluetooth module is embedded within the housing 1100 and serves as the central communication hub, enabling seamless data exchange between the device and the user's smartphone. When the weight sensor detects changes in the water bottle's weight, the thermometer measures the ambient air temperature, or the user interacts with the display screen, the Bluetooth module captures this data and transmits it in real-time to the paired software application. The wireless communication allows the data detected and input received into the application or display on housing to be synchronized between the display and the application. In some embodiments, a timer is integrated into the housing, wherein the timer is configured to track a duration between weight detection by the weight sensor.

In the illustrated embodiment, the hydration monitoring device and system 1000 further comprises notification system having a plurality of default voice and text notifications stored in the base 1200, which can be customized by the user via the application. The notifications are adapted to be triggered by real-time tracking of the hydration status via the weight sensor. The notification system comprises a target notification configured to alert the user if the change of the weight of the water is below a predetermined target rate and by how much below the predetermined target weight.

In one exemplary operation, the hydration monitoring device and system 1000 receives a hydration target. The hydration target is set in one of three ways. The first way is via a manual entry into the touch screen display or through the application. An example of a manual entry comprises entering the target water consumption in ounces, the number of hours to reach the target water consumption, and the frequency of notifications the user wishes to receive. The second way is to use a default setting and the third way is to allow the system to calculate the hydration target by receiving user profile information through the application. In one exemplified embodiment for the third way of setting the hydration target, the system calculates a baseline water consumption by multiplying the user's weight in pounds by a constant, such as 0.534 ounce/pound, resulting in the initial ounce measurement. A daily activity level is then factored in by adding a specific value based on the user's choice, ranging from zero to 48 ounces depending on the activity intensity (Light +6, Moderate +12, High +24, Very High +48). Any extra water needs, which can be entered by the user up to a maximum of 200 ounces, are also added to this total. These components are combined (a+b+c) to establish the hydration target.

Once the hydration target is set, the device calculates a hydration track, which delineates a rate of water consumption over a specified time period. In one exemplified embodiment, the hydration track is set by dividing the hydration target into day and night targets, with two-thirds allocated to the day and one-third to the night. The target rate for day and night is determined by further dividing these targets; the day target is split into 36 equal increments over 12 hours, while the night target is spread across a single 12-hour period. To track water intake, the weight of the water bottle is predetermined or tared by the device. As the user places the bottle into the holder, the device weighs the water, and this weight data is transmitted to the application. The application then translates the water weight into ounces and displays the quantity of water in the bottle on the display.

Users can start a 24-hour cycle with the application displaying the target amount, the amount consumed, the total water intake, and a timer. This allows users to monitor their hydration over the entire day. As the user drinks from the bottle, any change in weight is detected by the device, which updates the application in real time. The application then displays the updated target amount, the amount consumed, the total water intake, and a timer, clearly indicating the user's progress towards their hydration goals. This real-time monitoring feature ensures that users stay informed and motivated to maintain adequate hydration throughout the day.

If the user's consumption rate falls below the hydration target, the device issues notifications through both text and voice prompts, through the housing and display and/or the application. In the illustrated embodiment, the notification system delivers a notification or reminder one time per hour. A user can modify the frequency of receiving notifications and the types of messaging through the interface on the application. If the rate of consumption is below the target rate by more than 20%, the app sends a notification to the holder and the holder, through its speaker and screen, prompts the user to drink more. A decrease in weight is recorded as water consumption, added to amounts previously recorded for the day and compared to the desired rate of consumption. An increase in weight is recorded as a “refill.” Refill means that real time consumption for that fresh container of water is reset to zero. When water is removed from the refilled container, the amount of water consumed is added to the amounts previously consumed for the day.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A hydration monitoring device and system, comprising:
a housing configured to hold a water bottle in an in-use configuration;
a weight sensor disposed in the housing to detect a weight of the water bottle in the in-use configuration;
a display operably connected to the weight sensor;
a weight tracking system having a logic, that when executed by a processor causes water consumption to be tracked by measuring a change in the weight of the water bottle over time;
a notification system configured to provide visual notification to the display when a rate of water consumption falls below a hydration target.

2. The bottle holder with hydration monitoring system of claim 1, further comprising a handle extending from an exterior of the receptacle.

3. The hydration monitoring device and system of claim 1, wherein the housing comprises a substantially cylindrical shape.

4. The hydration monitoring device and system of claim 1, wherein the housing comprises a base removably securable to a receptacle.

5. The hydration monitoring device and system of claim 4, wherein the receptacle forms a sleeve when detached from the base.

6. The hydration monitoring device and system of claim 1, wherein the weight tracking system further comprises a timer integrated into the housing, wherein the timer is configured to track a duration between weight detections by the weight sensor.

7. The hydration monitoring device and system of claim 1, a thermometer integrated into the base to measure the ambient air temperature.

8. The hydration monitoring device and system of claim 1, further comprising a power source for powering the weight sensor and display.

9. The hydration monitoring device and system of claim 8, wherein the power source is a rechargeable battery, and wherein a charging port is disposed on the housing for charging the battery.

10. The hydration monitoring device and system of claim 1, wherein the display comprises an interface adapted to receive a user weight and user activity level to calculate a personalized hydration target.

11. The hydration monitoring device and system of claim 1, wherein the display is a touch screen.

12. The hydration monitoring device and system of claim 11, wherein the touch screen is adapted to manage settings of the device and display messages.

13. The hydration monitoring device and system of claim 1, further comprising a speaker disposed on the housing for delivering audible notifications to the user.

14. The hydration monitoring device and system of claim 1, further comprising a transceiver disposed within the housing for wireless communication with an application on an electronic device.

15. The hydration monitoring device and system of claim 14, wherein the application is configured to calculate a desired hydration rate based on a user profile data and activity levels.

16. The hydration monitoring device and system of claim 14, wherein the application comprises an application interface adapted to receive a user weight and user activity level to calculate a personalized hydration target.

17. The hydration monitoring device and system of claim 14, wherein the weight tracking system is synchronized between the display and the application.

18. The hydration monitoring device and system of claim 14, wherein the notification system comprises;
a plurality of default voice and text notifications stored in the base, which can be customized via the application;
wherein the notifications are triggered by real-time tracking of the user's hydration status via the weight sensor.

19. The hydration monitoring device and system of claim 1, wherein the hydration target is set via a manual entry into the display.

20. The hydration monitoring device and system of claim 1, wherein the manual entry comprises entering a target water consumption amount, a number of hours to reach the target water consumption amount, and a notification frequency
